(19) United States
(12) Patent Application Publication
(10) Pub. No.: US 2020/0294670 A1
(43) Pub. Date: Sep. 17, 2020

(54) SYSTEM AND METHOD FOR REAL-TIME ESTIMATION OF EMOTIONAL STATE OF USER

(71) Applicant: Monsoon Design Studios LLC, Los Gatos, CA (US)

(72) Inventors: Giuliana Kotikela, Los Gatos, CA (US); Aditya Sane, San Jose, CA (US); Daniel Housman, Newton, MA (US); Purav Gandhi, Ahmedabad (IN); Anshu Chittora, Ahmedabad (IN)

(73) Assignee: Monsoon Design Studios LLC, Los Gatos, CA (US)

(21) Appl. No.: 16/818,029

(22) Filed: Mar. 13, 2020

Related U.S. Application Data
(60) Provisional application No. 62/817,679, filed on Mar. 13, 2019.

(51) Int. Cl. G16H 50/20; G06N 20/10; G16H 40/60

(52) U.S. Cl. CPC G16H 50/20; G16H 40/60; G06N 20/10

(57) ABSTRACT

Various embodiments of the present disclosure provide a system and method for real-time estimation and changing emotional state of a user. The system comprises a memory configured to store executable instructions and a processor configured to execute the executable instructions stored in the memory, the processor configured to: collect bio physiological data of the user from a smart wearable device of the user, validate the bio physiological data, extract features based on the validated bio physiological data, and train a machine learning model using the extracted features for the real-time estimation of emotional state of the user, and provide personalized recommendations for changing the emotional state of the user.

### TECHNOLOGICAL FIELD

Various embodiments of the disclosure relate to a system and a method to determine an emotional and biophysiological state of a user. More particularly, the various embodiments of the present disclosure relate to a system and a method to accurately determine an emotional state of a user in real-time or near real-time for self-awareness, monitoring, and early intervention.

### BACKGROUND

Typically, a large number of people of all ages, genders and circumstances suffer from stress and anxiety, thereby leading to physical and psychological health issues in people. Stress may be defined as an uncomfortable emotional experience accompanied by predictable biochemical, physiological and behavioral changes. In certain scenarios, long working hours, less physical work, increase in virtual connections, and a lack of real communications are major contributors for the physical and psychological health issues in people.

Currently, Electrocardiography (ECG) is the gold standard to determine heart rate and associated cardiac biophysiological parameters to estimate an end user's emotional state. However, ECG signals require multiple leads (contact points) on different parts of the body of the user to acquire the electrical activity, which may be a challenging and intrusive task. In addition, ECG signals may present a serious limitation in terms of continuous measurement of heart rate in a passive manner due to the continuous monitoring of participants and subsequent analysis of physiological responses.

In some scenarios, active user involvement in data collection for psychological state estimation may introduce conscious and subconscious biases. By passively collecting and logging of biophysiological data and signals, unbiased results are classified real-time into emotional categories of an individual user. Classified results are coupled with the user's prior emotions and experiences may offer behavioral insights that drive self-discovery and introspection. In addition, the collection and cognition of biophysiological data may allow providers to tailor their services and offerings to create hyper-personalized experiences that may aid in cultivating seamless interactions between users and service providers. Accordingly, there is a need for a system and a method to simplify end user management and preemptive treatment of emotional conditions and risks associated with interpersonal behavior.

### SUMMARY

A system and a method are described to provide a system and a method to determine an emotional state of a user in real-time or near real-time for self-awareness and early intervention as shown in, and/or described in connection with, at least one of the figures, as set forth more completely in the claims.

These and other features and advantages of the present disclosure may be appreciated from a review of the following detailed description of the present disclosure, along with the accompanying figures in which like reference numerals refer to like parts throughout.

### BRIEF DESCRIPTION OF THE DRAWINGS

The embodiments of the invention are illustrated by way of example, and not by way of limitation, in the figures of the accompanying drawings in which:

FIG. 1 is a block diagram that illustrates a network environment of a system to determine an emotional state of a user in real-time or near real-time, in accordance with one embodiment; and

FIG. 2 is a flow chart that shows a processing pipeline for implementation of an exemplary method to determine an emotional state of a user in real-time or near real-time, in accordance with one embodiment.

### DETAILED DESCRIPTION OF DRAWINGS

The following described implementations may be found in the disclosed system and method to determine an emotional state of a user. Exemplary aspects of the disclosure may include a system to determine an emotional state of a user in real-time or near real-time.

The disclosed system may be configured to determine and estimate emotional state of a user in real-time or near real-time. Thereby, the system may encourage user introspection and self-discovery to align emotional goals with quantifiable outcomes. The societal impacts of the disclosed system and method may be easier end user management and preemptive treatment of emotional conditions and risks associated with interpersonal behavior that may allow self-awareness and early intervention.

The present invention may use machine learning models for the classification of emotions. The present invention may provide the end users with a real-time understanding of their biophysiological state (past and present) and correlation of such state with environmental stimuli, which enables the end users to be data informed, as the end users create and achieve emotional goals.

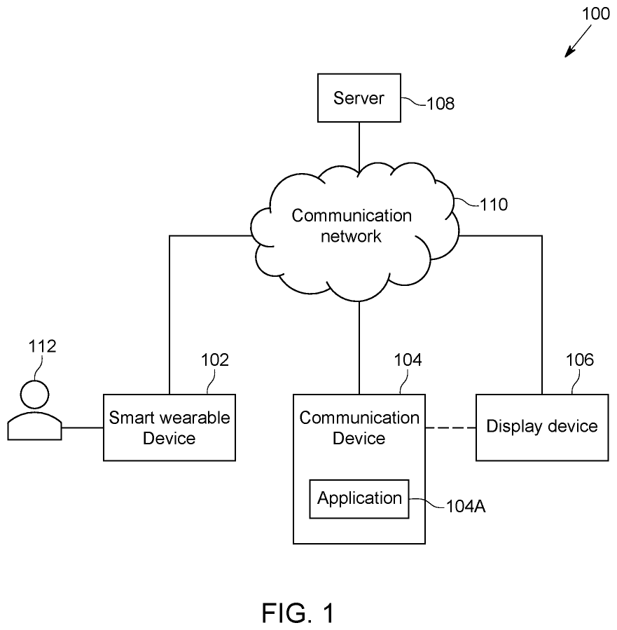

FIG. 1 is a block diagram that illustrates a network environment of a system to determine an emotional state of a user in real-time or near real-time, in accordance with one embodiment.

With reference to FIG. 1, there is shown a network environment 100 that may include a smart wearable device 102, a communication device 104, an application 104A associated with the communication device 104, a display device 106, a cloud server 108, and a communication network 110. The system may include the smart wearable device 102, the communication device 104 and the cloud server 108. A subject 112 may be associated with the smart wearable device 102. The subject 112 may also be associated with the communication device 104. The smart wearable device 102 may be communicatively coupled to the communication device 104, via the communication network 110. Further, the smart wearable device 102 may be communicatively coupled to the cloud server 108, via the communication network 110. Also, the smart wearable device 102 may be communicatively coupled to the display device 106, via the communication network 110. In accordance with an embodiment, the smart wearable device 102 may be directly coupled to the communication device 104. The smart wearable device 102 may also comprise a display screen (not shown in the FIG. 1).

The smart wearable device 102 may comprise suitable logic, circuitry, interfaces, and code that may be configured to monitor emotions of the subject 112 to assist the subject 112 in identification of the emotions and track the mood trajectory throughout any given day over time. The smart wearable device 102 may be configured to encompass a plurality of bio sensors for the measurement of biological signals. The smart wearable device 102 may be configured to use GPS in the smart wearable device 102. The smart wearable device 102 may be configured to use accelerometer to help the subject in tracking sleep and steps count. One or more of the aforesaid components of the wearable device 102 may be modular in structure and may be detachable from the wearable device 102.

The smart wearable device 102 may be configured to monitor heart rate of the subject 112 that may be recorded (in memory, such as cache memory) for a period of pre-defined days to track health related issues of the subject 112. The smart wearable device 102 may be configured to incorporate a microphone, such as a condenser mic, to record speech of the subject 112 along with bio signals of the subject 112. The smart wearable device 102 may comprise one or more Light Emitting Devices (LEDs) to provide feedback to the subject 112. The smart wearable device 102 may be configured to transmit alert notifications when the emotions damage fitness level of the subject 112 with personalized recommendations. In accordance with an embodiment, the smart wearable device 102 may perform the operations related to fitness applications along with emotional identification. The smart wearable device 102 may be configured to transmit notifications for a meeting in advance.

In accordance with an embodiment, the smart wearable device 102 may be studded with gems or design to give the smart wearable device 102 an appealing look suitable for all clothing, gender and ages. In accordance with an embodiment, the smart wearable device 102 may be configured to support wireless charging, such as induction charging. Typically, ECG signals may be used to identify the emotional state of a user, such as the subject 112 based on heart rate variability. However, Heart Rate Variability (HRV) signal which is a prime biological input for the emotional state classification may be extracted from a photoplethysmogram (PPG) signal using a sensor, such as a Sensor Probe for Pulse Oximetry (SPO2) sensor. The SPO2 sensor may be mounted on the smart wearable device 102. Examples of the smart wearable device 102 may include, but are not limited to, a ring, a smart jewelry, smart watch, and a smart band.

The communication device 104 may comprise suitable logic, circuitry, interfaces, and code that may be configured to receive biophysiological signals of the subject 112 from the smart wearable device 102. The communication device 104 may be configured to store the application 104A associated with the smart wearable device 102. The communication device 104 may be configured to provide geo-locational and prior emotional trajectory data to a prediction model stored in the cloud server 108. The communication device 104 may be configured with a plurality of sensors to collect various types of data, such as location data of the subject 112 who may be associated with the communication device 104. Examples of the communication device 104 may include, but are not limited to, mobile terminal, fixed terminal, or portable terminal including a mobile handset, station unit, device, multimedia tablet, Internet node, communicator, desktop computer, laptop computer, Personal Digital Assistants (PDAs), head-up display (HUD), augmented reality glasses, projectors, or any combination thereof. One or more users may be associated with the plurality of client devices 104A-104N.

The application 104A associated with the communication device 104 may help the subject 112 on understanding of the smart wearable device 102 and functionalities associated with the smart wearable device 102. The application 104A may be configured to provide a platform for the users, such as the subject 112 to give feedback on the smart wearable device 102. The application 104A may be configured to provide a platform for the users, such as the subject 112 to interact with a vendor of the smart wearable device 102 in case of trouble shooting. The application 104A may allow the subject 112 to analyze and track the biophysiological signals and emotional states over a pre-defined period. In accordance with an embodiment, the pre-defined period may correspond to, but not limited to, a day or 10 days. In accordance with an embodiment, a website may also be used that may be associated with the smart wearable device 102.

The application 104A may allow the subject 112 to keep a track of the variations in emotional data stored in an emotional database. The emotional data may allow the subject 112 to capture the history to be used as and when needed. The emotional data may help in special cases, such as psychological disorders to assess and sort the treatment. In accordance with an embodiment, the emotional database associated with the application 104A may be stored in cache memory of the communication device 104. In accordance with an embodiment, the emotional database associated with the application 104A may be stored in the cloud server 108. The application 104A may be configured to convert output of the prediction model in user friendly information and instructions.

The display device 106 may comprise suitable logic, circuitry, interfaces, and code that may be configured to provide user interface element on the communication device 104. The display device 106 may be communicatively coupled to the communication device 104, via the communication network 110. In accordance with an embodiment, the display device 106 may be a part of the communication device 104.

The cloud server 108 may comprise suitable logic, circuitry, interfaces, and code that may be configured to train a classifier model. The classifier may have a support vector machine classifier or Gaussian Mixture model (GMM) classifier or neural networks. The classifier parameters such as kernel function and their parameters in SVM and the type of neural networks and their kernel functions may be varied to find the best possible choice of the classifier model. The performance of the possible choice of classifier may be checked by applying the validation data set on the classifier and evaluating their performance using sensitivity and selectivity parameters. The cloud server 108 may be configured to train machine learning model in classification of emotions using extracted features.

In accordance with an embodiment, the cloud server 108 may be configured to interact with the application 104A, via the communication network 110. Such interaction may be based on an authentication received from the communication device 104. The cloud server 108 may be implemented using several technologies that are well known to those skilled in the art.

It may be appreciated that the cloud server 108 illustrated in FIG. 1 may be deployed in other ways and that the operations performed and/or the services provided by such servers may be combined or separated for a given implementation and may be performed by a greater number or fewer number of servers. One or more servers may be operated and/or maintained by the same or different entities. The data from the cloud server 108 may be ingested and processed by the communication device 104.

The communication network 110 may comprise suitable logic, circuitry, and interfaces that may be configured to provide a plurality of network ports and a plurality of communication channels for transmission and reception of data, such as visual data, location data, biophysiological data. Each network port may correspond to a virtual address (or a physical machine address) for transmission and reception of the communication data. For example, the virtual address may be an Internet Protocol Version 4 (IPv4) (or an IPv6 address) and the physical address may be a Media Access Control (MAC) address. The communication network 110 may include a medium through which the smart wearable device 102, the communication device 104 and/or the cloud server 108 may communicate with each other. The communication network 110 may be associated with an application layer for implementation of communication protocols based on one or more communication requests from at least one of the one or more communication devices. The communication data may be transmitted or received, via the communication protocols. Examples of such wired and wireless communication protocols may include, but are not limited to, Transmission Control Protocol and Internet Protocol (TCP/IP), User Datagram Protocol (UDP), Hypertext Transfer Protocol (HTTP), File Transfer Protocol (FTP), ZigBee, EDGE, infrared (IR), IEEE 802.11, 802.16, cellular communication protocols, and/or Bluetooth (BT) communication protocols.

Examples of the communication network 110 may include, but is not limited to a wireless channel, a wired channel, a combination of wireless and wired channel thereof. The wireless or wired channel may be associated with a network standard which may be defined by one of a Local Area Network (LAN), a Personal Area Network (PAN), a Wireless Local Area Network (WLAN), a Wireless Sensor Network (WSN), Wireless Area Network (WAN), Wireless Wide Area Network (WWAN), a Long Term Evolution (LTE) network, a plain old telephone service (POTS), and a Metropolitan Area Network (MAN). Additionally, the wired channel may be selected on the basis of bandwidth criteria. For example, an optical fiber channel may be used for a high bandwidth communication. Further, a coaxial cable-based or Ethernet-based communication channel may be used for moderate bandwidth communication.

In operation, the subject 112 may wear the smart wearable device 102 to estimate the emotional state in real-time or near real-time. The estimation of the emotional state of the subject 112 may help in certain cases to improve or influence the behavior pattern of the subject 112.

The smart wearable device 102 may encapsulate a plurality of biophysiological sensors, such as plethysmography (PPG), galvanic skin response (GSR), skin temperature, electrocardiogram (ECG), and ambient audio. In accordance with an embodiment, the PPG, GSR, ECG, and skin temperature may be placed on the interior surface of the smart wearable device 102. In accordance with an embodiment, the ambient audio sensor is may be placed on the exterior surface. The smart wearable device 102 may be composed of at least two segments. The mold die cast metal based top segment that may be hollowed to accommodate the plurality of sensors. The lower segment of the smart wearable device 102 may be composed of a composite material that is flexible and may have elastic properties. To allow for minor adjustments in the size of the smart wearable device 102. The lower segment of the smart wearable device 102 may provide a snug fit of the biophysiological sensors from an interior surface of the smart wearable device 102 to the skin of the subject 112. The two segments of the smart wearable device 102 may be connected using an interlock joint hinge. The smart wearable device 102 may be configured to incorporate rechargeable power source that can rectify mains power supply. The smart wearable device 102 may have at least a microprocessor and volatile memory to cache the biophysiological signals. The microprocessor of the smart wearable device 102 may be configured to communicate with the application 104A. In accordance with an embodiment, the microprocessor of the smart wearable device 102 may be communicatively coupled to the communication device 104 through Bluetooth technologies as defined by standard IEEE 802.15.1, via the communication network 110.

The smart wearable device 102 may be comprised of a plurality of biophysiological sensors, at least the microprocessor, at least a non-volatile memory, at least a power supply, and one or more wireless components that may be placed into malleable surface of the smart wearable device 102. In accordance with an embodiment, the top interior surface of the smart wearable device 102 may have a display unit. In accordance with an embodiment, the display unit of the smart wearable device 102 may be composed of a turbid sapphire crystal that may be illuminated by a multi-colored LED.

The smart wearable device 102 may be configured to incorporate the microphone, such as a condenser mic, to record speech of the subject 112 along with bio signals of the subject 112. In accordance with an embodiment, the speech of the subject 112 may vary with the varying moods of the subject 112. For example, voice is raised in angry mood and voice is lowered in depressed mood by a person.

The smart wearable device 102 may be configured to generate PPG signal that along with additional sensor input, such as skin temperature may be used for electrode calculation. The ECG signals may be replaced with the PPG signals because ECG and PPG signal patterns correlate that allows usage of PPG signals to estimate emotional state of the subject 112.

The communication device 104 may receive data related to the bio signals from the smart wearable device 102 via the communication network 110. The application 104A of the communication device 104 may be configured to process data associated with the smart wearable device 102. The application 104A may be configured to cache and consume biophysiological data from the smart wearable device 102 and the communication device 104. The application 104A of the communication device 104 may be configured to transform the biophysiological data for uploading to the cloud server 108 for emotional state estimation. The application 104A may have user interface elements (UIs) such that the subject 112 may search and access historical biophysiological data.

The application 104A may be configured to display emotional state of the subject on the display device 106 associated with the communication device 104. The current and historical emotional states of the subject 112 may be displayed through an intuitive user interface that may be linked to the smart wearable device 102. In accordance with an embodiment, the subject 112 may perform trend analysis to determine potential correlations with external stimuli. The application 104A may be configured to aggregate population level emotional states that may be aligned to a profile of individual user and to access market segment.

The application 104A may be configured to provide security and access control to the subject 112. An application level access may be authenticated through an application programming interface (API). The application 104A may be configured to encrypt data on the communication device 104. For example, the data on the communication device 104 may be encrypted by using a FIPS 140-2 cypher. The application 104A may be configured to transport data from the smart wearable device 102 to the cloud server 108. The transported data may be encrypted. For example, the data may be encrypted through a HTTPS transport. The application 104A may be configured to delegate user emotional state and profile to a third party entity or user.

The application 104A may be configured to analyze data related to emotional state of the subject 112 with the help of classifier and prediction model on the cloud server 108. The application 104A may be configured to provide temporal analysis of previous events that potentially trigger outliers in an emotional state of the subject 112. The application 104A may be further configured to provide geospatial analysis to identify locations that may be correlated with outliers in emotional state of the subject 112. The application 104A may be configured to identify locations and experiences of different users belonging to the same market segment. The application 104A may be configured to identify emotional state of users who opted-in to share the emotional data with a third party vendor or individual user.

The cloud server 108 may be configured to provide infrastructure to store historical biophysiological data from all users (such as the subject 112). The cloud server 108 may be configured to use a plurality of layers, such as data sources, data acquisition and integration, data storage and processing, business semantic layer, information delivery, information consumers/processors, data governance and standards. The cloud server 108 may be configured to transform biophysiological data into a set of relevant features. The cloud server 108 may be configured to estimate emotional state of the subject 112 based on the set of relevant features in a prediction model. The cloud server 108 may be configured to train the prediction model from an experimental trial for initial calibration for estimation of emotional state of the subject 112. The cloud server 108 may be configured to establish user specific baselines for calibration based on initialization of the smart wearable device 102. A user-level baseline may be determined during the initialization of the smart wearable device 102 for the subject 112. During the initialization, signals captured may be compared against calibration data and adjustments may be made to coefficients and parameters. The application may be tuned according to a user, such as the subject 112. Therefore, the application 104A may continue to generate emotion estimates for each tranche or window of data captured. While the user-level baseline adjustments may be done using the cloud server 108, the calculation of emotional state may be done within the application on the communication device 104.

The application 104A may be configured to gather data from the subject 112. However, because of the sensitive nature of the data, the data may be securely backed up to the cloud server 108. Additional details about emotional state and user level trends may be provided within the application 104A. The data may be analyzed through the prediction model in the cloud server 108. The prediction model may provide insight for the subject 112 to analyze emotional trends, co-ocurrences, and other attributes of interest. The application 104A may be configured to receive feedback on the emotional states and stimuli from the users, such as the subject 112 based on user log in to the application 104A. The prediction model may be further refined based on user input through the application 104A for further unsupervised learning. The cloud server 108 may be configured to anonymize the data and aggregate the data to provide a population or community view of emotional states and wellbeing of the subject 112.

The smart wearable device 102 may be configured to obtain biophysiological signals of the subject 112 after a predefined time interval. The smart wearable device 102 may be configured to cache the obtained biophysiological signals of the subject 112. The smart wearable device 102 may be configured to transfer the biophysiological signals of the subject 112 to the communication device 104 immediately, if available, or keep on the smart wearable device 102 for not less than a predefined time. The communication device 104 may be configured with a secondary cache that may be synchronized with the cloud server 108. The synchronization of the communication device 104 with the cloud server may be either immediate or when the communication network 110 is available. The cloud server 108 may be intended to persist the user level biophysiological signals for a long term, such as for at least one year or longer. User feedback of classified emotional states may be provided through a multi-color LED display on the smart wearable device 102. User level trends may be useful for understanding periodically occurring external stimuli. For example, user stress levels during daily commute, and identification or recommendations of alternative routes based on the users happy path. When initializing the ring for the first time, a user calibration will occur in order to generate a baseline of the user emotional state. Subsequent analysis will use the established baseline to monitor and assess activities progress and effectiveness for the subject 112.

The sensor data from the smart wearable device 102 may be cached on the smart wearable device 102 in the cache memory and transmitted to the communication device 104 using Bluetooth technology as defined in IEEE Standard 802.15.2. The sensor data in the communication device 104 may be used by the application 104A. The application 104A may assign different weights to each of the sensor data for further processing by the application 104A and/or the cloud server 108. The sensor data may be integrated with geospatial location (using GPS) and accelerometer data from the communication device 104. The integrated data is transferred to the cloud server 108 for subsequent analysis. Responses from the cloud server 108 may be displayed on the communication device 104 and a limited set of user feedback provided with the smart wearable device 102 mounted multi-colored LEDs.

The smart wearable device 102 may provide to the subject 112 automatic and passive log emotional states and locations. The subject 112 may understand and respond to a stimulus without having to respond to surveys or reviews. The emotional response estimated from biophysiological sensors may be a true representation of the experience that may be used for personal analysis or shared with the community. The emotional responses may be hyper personalized for services and offerings per user.

The system may allow the subject 112 to monitor mood swings and suggests activities according to the mood of the subject 112. The smart wearable device 102 may be a low cost and attractive device and small in size. In accordance with an embodiment, children and elderly people may help the guardians in caring and parenting with the smart wearable device 102. The LED mood lights of the smart wearable device 102 may change color with mood, and allows visual indications to the subject 112. The communication device 104 that may be interfaced with the smart wearable device 102 may provide recommendations to the subject 112 as per the detected mood of the subject 112.

The system may be configured to use machine learning methods for prediction model for classification of emotions. The machine learning methods may help in various real life applications such as related to speech processing, image processing and natural language processing. The cloud server may be configured to derive features from various methods that may be used alone or combined with score level methods or feature level fusion methods. The features may be used to train a classier. The classifier may have a support vector machine classifier or Gaussian Mixture model (GMM) classifier or neural networks. The selection of the final classifier will be based on their ability to classify the signals in correct emotion class. The classifier parameters such as kernel function and their parameters in SVM and the type of neural networks and their kernel functions will be varied to find the best possible choice of the classifier model. The performance of the possible choice of classifier will be checked by applying the validation data set on the classifier and evaluating their performance using sensitivity and selectivity parameters.

The communication device 104 may be configured to use the prediction model built using training dataset. The prediction model may be used to classify the bio signals to classify the bio signals to corresponding emotion of the subject 112. The application in the communication device 104 will extract features from the bio signals and then these features are applied to a classifier model which classifies the incoming features in one of the classes which has the highest probability/minimum distance from available statistical models. For this purpose, the classifier model may utilize the weights assigned to the sensor data (incoming features). Once the decision regarding a possible emotional state of the subject 112 may be made by the application 104A from the prediction model from the cloud server 108, the application 104A may be configured to check options that may help in improving the mood of the subject 112. For example, the application 104A may suggest the subject 112 to take a nap, listen to favorite music, or go for a walk. The suggestions by the application 104A may be based on the emotions identified according to the GPS location of the subject, For example, the subject 112 may be happy while visiting a nearby market, the application 104A may advise to go for a shopping.

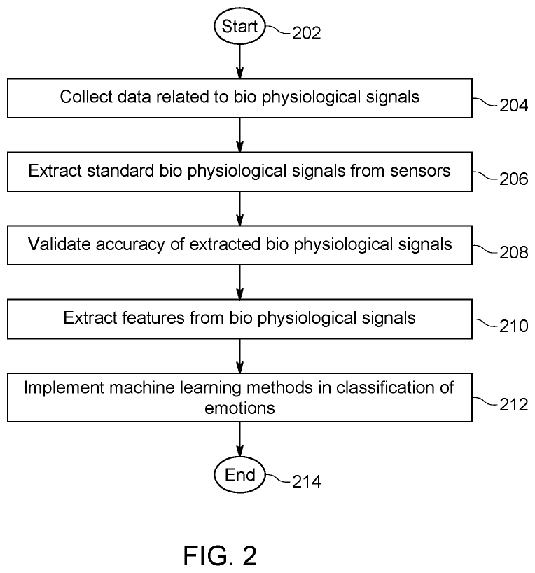

FIG. 2 is a flow chart that shows a processing pipeline for implementation of an exemplary method to determine an emotional state of a user in real-time or near real-time, in accordance with one embodiment.

With reference to FIG. 2, there is shown a flowchart 200. The flowchart 200 is described in conjunction with elements from FIG. 1. The method, in accordance with the flowchart 200, may be implemented in the system that comprises smart wearable device 102, the communication device 104, the display device 106 and the cloud server 108. The method starts at 202 and proceeds to 204. The method may aid in analyzing the bio signals collected over a period to identify the emotional well-being of a user, such as the subject 112 and provide recommendations and alerts.

At 204, data related to biophysiological signals (bio signals) may be collected. The data may be collected from the smart wearable device 102 to calibrate the system. The data collected may be divided in train, test and validation sets. The validation dataset may be used to test classifier performance and validation of the classifier. Further, with initial samples of the subject 112, the method for prediction of the emotional state may be validated. In accordance with an embodiment, modifications may be made by the system based on the samples of the subject 112.

The data may be collected under simulated conditions from a focus user group. In accordance with an embodiment, the group may comprise of large number of human subjects that are exposed to numerous audio-visual stimuli intended to invoke specific emotional responses over a specific time period. The data for the method may be collected from various subjects who are healthy and satisfy the age criteria. In accordance with an embodiment, the bio signals may be collected from different sites to include the regional variability.

For data collection, the smart wearable device 102 may be composed of a plurality of sensors. In accordance with an embodiment, the sensors may be placed in a wearable belt of the smart wearable device 102 that may be tightened with a velcro. In accordance with an embodiment, the sensor belt may be worn on a finger (such as, ring finger) to collect the bio signals. The smart wearable device 102 may be configured to capture the biophysiological signals using a prototype device of the smart wearable device 102 along with ancillary signals such as electrocardiogram (ECG) from an FDA-cleared instrument. The ancillary signals may be required to establish design-time correlation between the biophysiological signals of the smart wearable device 102 and a known standard. The sensor belt of the smart wearable device 102 may comprise an ECG sensor, SPO2 sensor, and GSR sensor that may record the heart rate, PPG signal and skin conductance. The cohort may be enrolled to match the initial target population demographics.

The cloud server 108 may be configured to receive calibration data for further analysis of the different emotions. Variability in the bio signals may be caused by different moods of the subjects. The subjects, such as the subject 112 may be shown videos related to different emotions in order to elicit an emotion and thereby changes in the corresponding biological signal may be noted. The emotions may correspond to, but not limited to, anger, fear, pleasure, sad, disgust and thrill. The emotional states, such as, happy and excited may be termed as positive while anger and sad states may be termed as negative emotional states. In accordance with an embodiment, after showing the video of a particular emotion, there will be display of neutral images to nullify the effect of the earlier video on the emotional state of the subjects. The calibration data may be analyzed for identification of the appropriate supervised and unsupervised learning models. Based on the identification of the appropriate supervised and unsupervised learning models, signal pre-processing, feature generation, and prediction model may be selected. Coefficients and parameters for the prediction model may also be calculated at the cloud server 106. The calibration of data may be verified using a hold-out dataset. Subsequent data may be captured from a smaller different cohort that is statistically equivalent to the original cohort. The calibration of the data may be done offline using a cloud server 108.

At 206, standard biophysiological signals may be extracted from sensors present in the smart wearable device 102. In accordance with an embodiment, SPO2 sensor of the smart wearable device 102 may be configured to find heart rate variability (HRV) and heart rate. PPG signal from the SPO2 sensor of the smart wearable device 102 may be used to determine heart rate and heart rate variability because the PPG signal may be collected from any part of the body where capillaries are found close to the skin surface of the subject 112. Moreover, no application of gel or any adhesive may be required for the PPG signals by the smart wearable device 102. A peak to peak interval may be calculated by calculating the difference between two peaks of a first derivative of the PPG signals. The peak to peak interval may correspond to RR interval in ECG signal which in turn measures the heart rate in PPM.

The heart rate variability may be defined as the power spectral density between two R-R peaks in an ECG signal. Similarly, the heart rate variability may be defined in PPG derivative signals as the corresponding peaks and the time series between them may be used to determine the HRV signal that may be called the PPI signal. Initially, the PPI signal may be interpolated using cubic spline method, in accordance with an embodiment. The resulting signal may be mean subtracted to remove the DC signal and then the signal is high pass filtered through a cut off frequency of 0.03 Hz to eliminate the low frequency disturbances. The signal may correspond to the equivalent HRV signal and analyzed using Fast Fourier Transform (FFT) method to find power in different frequency bands.

At 208, accuracy of extracted biophysiological signals may be validated. In accordance with an embodiment, the cloud server 108 may be configured to validate the extracted biophysiological signals. During data collection, the smart wearable device 102 may also incorporate ECG sensor along with SPO2 sensor. The ECG sensor may be configured to validate the extraction of heart rate variability and heart rate from the SPO2 sensor output. The same features extracted from the ECG and PPG signals may be compared for the variations and differences in the mean and standard deviations. Based on verification of the PPG signal to be used as an alternative to the ECG signal, the SPO2 sensor may be used in the smart wearable device 102. In accordance with an embodiment, the equivalence of ECG and PPG signals may be validated based on evaluation of classification by features derived from ECG and PPG signals. The ECG signal may be replaced with the PPG signal based on classification accuracy and other statistical measures of the classification.

At 210, features from biophysiological signals may be extracted. In accordance with an embodiment, the cloud server 108 may be configured to extract the features. The features may be extracted to identify different emotions from the bio signals. Additional features and spectral transformations are done at the cloud server 108 to determine features that may be used in classification of emotional state of the subject 112.

The data from the biophysiological sensors of the smart wearable device 102 may be pre-processed to remove noise, motion artifacts, point discontinuities, jump discontinuities, point outliers, and other effects that can cause errors in emotion classification. The cloud server 108 may be configured to preprocess the data from biophysiological sensors. In accordance with an embodiment, the data may also be calculated through a window that can be normalized using a Hamming or a Hanning function. In accordance with an embodiment, the data may further be transformed into the frequency domain through the use of a Fourier transform or a Wavelet transform. The data may be developed into features that are used for machine learning to estimate emotional state.

The bio signals collected from the smart wearable device 102 are multiple. However, for classification of the emotions, suitable features may be extracted from the bio signals. The bio signals may be filtered. The filtered signals may be processed to extract the features from the heart rate variability signal extracted from the PPG signal. The features extracted may correspond to, but not limited to, the coefficients of the Fast Fourier transform, wavelet transform, and power in different energy bands. Statistical parameters may be extracted from the HRV signal over a fixed period (say every 10 seconds). The statistical parameter may correspond to mean, variance, standard deviation, kurtosis, skewness, maximum response, proportion of negative samples in the derivative versus all samples of PPG signal and skin conductance. In accordance with an embodiment, non-linear signal processing techniques such as using fractals, higher order spectra analysis, and turbulence analysis may also be used for signal processing. In accordance with an embodiment, feature selection or dimension reduction methods like PCA, ICA may be applied to determine the important features and thereby the sensors. Therefore redundant features may be avoided. As a result, feature size may be reduced and classification time may be reduced. Recognizing a suitable classifier which gives high classification accuracy and requires less time and space may be done through rigorous experiments. Various classifier models and features may be used to classify the emotions. The classifier that satisfies the criteria of computation time, memory and accuracy may be used in final analysis and corresponding feature set will be used in feature extraction.

In accordance with an embodiment, feature extraction may have some preprocessing of the signals like filtering and amplitude normalization. some of the features may include power spectral density estimation in different frequency bands, differences in energy between different frequency bands, correlation analysis, linear transformation using linear signal processing techniques and non-linear methods for feature extraction like finding fractals, higher order spectral analysis using bispectrum or trispectrum.

Features derived from various methods will be used alone or combined using score level or feature level fusion methods. The features may be used to train a classier. The classifier may have a support vector machine classifier or Gaussian Mixture model (GMM) classifier or neural networks. The selection of the final classifier will be based on their ability to classify the signals in correct emotion class. The classifier parameters, such as kernel function and their parameters in SVM and the type of neural networks and their kernel functions will be varied to find the best possible choice of the classifier model. The performance of the possible choice of classifier will be checked by applying the validation data set on the classifier and evaluating their performance using sensitivity and selectivity parameters.

At 212, machine learning methods may be implemented in classification of emotions of a user, such as the subject 112. The machine learning methods may be implemented in the cloud server 108. In accordance with an embodiment, machine learning may be used to estimate the emotional state of the subject 112 based on a combination of biophysiological signals as a proxy for ECG. The machine learning methods may comprise unsupervised and supervised learning. A machine learning tool such as support vector machine, Gaussian mixture model classifier (GMM), neural networks, or k-nearest neighbor classifier may be used to train the prediction model from the extracted features. The signals may be classified in different emotional states, based on the trained prediction model. In accordance with an embodiment, a combination of the classifiers may be used to improve the classification of emotions. The baseline may be calibrated by machine learning methods for overall population and individual users. Machine learning methods, such as, but not limited to, regression, classification, neural networks, support vector machines, may be used in biometric and biophysiological applications.

The smart wearable device 102 may be communicatively coupled with the communication device 104 to connect the smart wearable device 102 ring with the subject 112. The bio signals from the smart wearable device 102 may be transmitted to the communication device 104 using a communication network, such as Bluetooth technology. The bio signals may be received at the communication device 104. The application 104A may receive the bio signals. The bio signals may be used to classify the emotion of the subject 112 in any one of the possible mood based on training of the prediction model. The application 104A may be integrated with the classifier model and the trained prediction model available on the cloud server 108. The bio signals received from the smart wearable device 102 may be applied to the classifier model and then classified into one of the emotion. Based on the decision of the classifier, the application will recommend few things to change. For example, in case of a joyful mood, the application 104A may be configured to suggest playing an outdoor game or suggesting visiting a nearby newly opened hotel. In accordance with an embodiment, the application 104A may be configured to suggest automatically changing the playlist to party songs or advising to book a drama in a theater.

Users, such as the subject 112 may be interested in understanding emotional state and wellbeing in response to external stimuli to achieve personal wellness goals and happiness. The stimuli may include food, sights, sound, location, people and tactile objects. The disclosed system and method may alter the environment, interpersonal interactions, and response to stimuli of the subject 112 based on an awareness of prior emotional responses. The responses may be at the individual or aggregated at the population level. In accordance with an embodiment, service providers may tailor the offerings to generate hyper-personalized experiences for existing and identification of new customers.

The method may be a per-user method to consolidate of data sourced from multiple biophysical sensors, geographic information sensors, and prior emotional and experiential responses. The method may calculate, calibrate, and correlate the expected emotional and experiential responses through the use of predictive modeling methods. The method may use grouped data to estimate the expected emotional and experiential responses.

The present disclosure may be realized in hardware, or a combination of hardware and software. The present disclosure may be realized in a centralized fashion, in at least one computer system, or in a distributed fashion, where different elements may be spread across several interconnected computer systems. A computer system or other apparatus adapted to carry out the methods described herein may be suited. A combination of hardware and software may be a general-purpose computer system with a computer program that, when loaded and executed, may control the computer system such that it carries out the methods described herein. The present disclosure may be realized in hardware that comprises a portion of an integrated circuit that also performs other functions.

The system and method may passively collect biophysical signals from the smart wearable device 102 wearable coupled with prior emotions and experiences to determine unbiased emotional needs, wants, and desires of the subject 112. Therefore, the subject 112 may automatically log into actual experiences anytime and anyplace. In accordance with an embodiment, the system may be configured to provide personalized categorical recommendations, without the bias and burden of writing manual reviews that typically generate inflated and irrelevant feedback.

The present disclosure may also be embedded in a computer program product, which comprises all the features that enable the implementation of the methods described herein, and which when loaded in a computer system is able to carry out these methods. Computer program, in the present context, means any expression, in any language, code or notation, of a set of instructions intended to cause a system that has an information processing capability to perform a particular function either directly, or after either or both of the following: a) conversion to another language, code or notation; b) reproduction in a different material form.

While the present disclosure has been described with reference to certain embodiments, it will be understood by those skilled in the art that various changes may be made and equivalents may be substituted without departure from the scope of the present disclosure. In addition, many modifications may be made to adapt a particular situation or material to the teachings of the present disclosure without departure from its scope. Therefore, it is intended that the present disclosure not be limited to the particular embodiment disclosed, but that the present disclosure will include all embodiments that falls within the scope of the appended claims.

1. A system for real-time estimation and changing emotional state of a user, the system comprising:
a memory configured to store executable instructions; and
a processor configured to execute the executable instructions stored in the memory, the processor configured to:
collect bio physiological data of the user from a smart wearable device of the user:
validate the bio physiological data;
extract features based on the validated bio physiological data:
train a machine learning model using the extracted features for the real-time estimation of emotional state of the user; and
change emotional state of the user based on the estimation.

2. The system of claim 1, wherein the processor is further configured to perform one or more of:
plethysmography (PPG);
Galvanic Skin Response (GSR);
monitor heart rate and a heart rate variability (HRV) signal of the user;
collect speech of the user;
provide feedback to the user using one or more light emitting devices (LEDs); and
transmit notifications or alerts with personalized recommendations to the user, the personalized recommendations or alerts are generated based on the emotional state of the user.

3. The system of claim 1, wherein the processor is configured to enable the user to change from current emotional state to different emotional state in real-time based on the estimated emotional state of the user.

4. The system of claim 2, wherein the personalized recommendations or alerts are provided to change the emotional state of the user from current emotional state to different emotional state.

5. The system of claim 1, wherein the processor is configured to provide geo-locational and emotional trajectory data of the user to the machine learning model.

6. The system of claim 5, wherein the machine learning model comprises a classifier model, generalized regression, non-linear regression, the classifier model further comprises one or more of:
a support vector machine (SVM), a Gaussian Mixture model (GMM), a k-nearest neighbour classifier, and a neural network classifier.

7. The system of claim 1, wherein the processor is configured to synchronize a cache of the smart wearable device, the cache comprises the bio physiological data of the user.

8. The system of claim 1, wherein the processor is further configured to enable the user to analyse and track the bio physiological data and emotional states of the user for a pre-defined time period using an application.

9. The system of claim 8, wherein the application is configured to:
gather data associated with user level trends of the user; and
receive feedback on the emotional state and external stimuli from the user.

10. The system of claim 1, wherein the machine learning model is trained from an experimental trial for initial calibration for the estimation of the emotional state of the user.

11. The system of claim 10, wherein the processors is configured to establish user specific baselines for the initial calibration based on initialization of the smart wearable device.

12. The system of claim 11, wherein the processor is configured to perform data anonymization of the bio physiological data and aggregate the bio physiological data to provide a population view of the emotional state and well-being of the user.

13. The system of claim 12, wherein the processor is further configured to store historical bio physiological data of the user.

14. The system of claim 1, wherein the processor is configured to process the machine learning model in one or more of: the smart wearable device, a communication device, and a cloud server.

15. A computer-implemented method for real-time estimation and changing emotional state of user, the computer-implemented method comprising:
collecting bio physiological data of the user from a smart wearable device of the user;
validating the bio physiological data;
extracting features based on the validated bio physiological data;
training a machine learning model using the extracted features for the real-time estimation of emotional state of the user; and
changing emotional state of the user based on the estimation.

16. The computer-implemented method of claim 15, further comprising:
plethysmography (PPG);
Galvanic Skin Response (GSR);
monitor heart rate and a heart rate variability (HRV) signal of the user;
collect speech of the user;
provide feedback to the user using one or more light emitting devices (LEDs); and
transmit notifications or alerts with personalized recommendations to the user, the personalized recommendations or alerts are generated based on the emotional state of the user.

17. The computer-implemented method of claim 15 further comprising enabling the user to change from current emotional state to different emotional state in real-time based on the estimated emotional state of the user.

18. The computer-implemented method of claim 16, wherein the personalized recommendations or alerts are provided to change the emotional state of the user from current emotional state to different emotional state.

19. The computer-implemented method of claim 15, further comprising providing geo-locational and emotional trajectory data of the user to the machine learning model.

20. The computer-implemented method of claim 15, further comprising enabling the user to analyze and track the bio physiological data and emotional states of the user for a pre-defined time period using an application.

21. The computer-implemented method of claim 20, further comprising gathering data associated with user level trends of the user and receiving feedback on the emotional state and external stimuli from the user, via the application.

22. The computer-implemented method of claim 15, wherein the machine learning model is trained from an experimental trial for initial calibration for the estimation of the emotional state of the user.

23. The computer-implemented method of claim 22, further comprising establishing user specific baselines for the initial calibration based on initialization of the smart wearable device.

24. The computer-implemented method of claim 23, further comprising performing data anonymization of the bio physiological data and aggregation of the bio physiological data to provide a population view of the emotional state and well-being of the user.

25. The computer-implemented method of claim 24, further comprising storing historical bio physiological data from the user.

26. The computer-implemented method of claim 15, further comprising processing the machine learning model in one or more of: the smart wearable device, a communication device, and a cloud server.
